(19) United States
(12) Patent Application Publication
(10) Pub. No.: US 2007/0184129 A1
(43) Pub. Date: Aug. 9, 2007

(54) Ginseng composition for preventing or improving the lowering of concentration and memory capability

(75) Inventors: Sung-Sick Woo, Seoul (KR); Ji-Min Cha, Seoul (KR); Dong-Seon Kim, Daejeon (KR); Sun-Young Sung, Gyeonggi-do (KR); Seon-Gli Do, Chungcheongbuk (KR); Young-Chul Lee, Daejeon (KR); Il-Hyoung Jung, Busan (KR); Young-Moon Heo, Jeollabuk-do (KR); Ju-Yeon Lee, Daegu (KR); Mi-Sun Oh, Chungcheongnam-do (KR)

Correspondence Address: FITZPATRICK CELLA HARPER & SCINTO, 30 ROCKEFELLER PLAZA, NEW YORK, NY, 10112, US

(73) Assignee: UNIGEN INC., Byeongcheon-myeon (KR)

(21) Appl. No.: 11/629,092

(22) PCT Filed: Jun. 10, 2005
(86) PCT No.: PCT/KR05/01764

§ 371 Date: Dec. 11, 2006

(30) Foreign Application Priority Data
Jun. 11, 2004 (KR) 10-2004-0043014

(51) Int. Cl. A61K 36/254

(52) U.S. Cl. 424/728

(57) ABSTRACT

The present invention relates to a ginseng composition equally comprising more than a certain amount of 10 kinds of ginsenoside in ginseng having particular pharmacological effect; or a composition for the prevention or improvement of lowering of concentration and memory capability comprising ginsenoside Rg3, Rg5 or RkI; a use thereof; a method for the prevention or improvement of lowering of concentration and memory capability by administrating the above composition; and a method for preparing agents for the prevention or improvement of lowering of concentration and memory capability by extracting ginseng etc. or mixing 10 kinds of ginsenoside in ginseng.

### TECHNICAL FIELD

The present invention relates to a ginseng composition equally comprising more than a certain amount of 10 kinds of ginsenoside in ginseng having particular pharmacological effect; or a composition for the prevention or improvement of lowering of concentration and memory capability comprising ginsenoside Rg3, Rg5 or Rk1; a use thereof; a method for the prevention or improvement of lowering of concentration and memory capability by administrating the above composition; and a method for preparing agents for the prevention or improvement of lowering of concentration and memory capability by extracting ginseng etc. or mixing 10 kinds of ginsenoside in ginseng.

### BACKGROUND ART

As the social environment is diversified, people come in contact with more stimuli, and rapid brain activity is needed to accept such stimuli. Also, aging of the population due to improvement of the living standard and development of the science increases chronic degenerative diseases such as memory loss from hypofunction of central nerves, amnesia, increase of uneasiness, etc. The prevention and treatment of dementia related to brain function among said chronic degenerative diseases of the old have become an important issue. Also, it is our reality that early education for children as well as teenagers is overheated due to fierce competition for college entrance examination. Under the reality, with private education fever, various programs regarding study and memory improvement have been introduced. In particular, some products developed for learning and memory improvement, and advertised that they can prevent dementia of the old and improve learning ability of teenagers.

Up to the present, some medicines and functional food using Chinese medicine formulas have been developed as goods for memory improvement and activation of brain function, but their effects have not been clearly proven.

On the other hand, according to Encyclopedia of Chinese Medicine, “ginseng stimulates the mental function by activating brain activity, and improves vision, hearing, intelligence, memory, and concentration.” Also, Shinnong-boncho-kyung says that “ginseng stabilizes the mind and enables people to be open-hearted and wiser.” In fact, some experimental results show that ginseng strengthens the brain activity, and improves learning activity, memory, and action. Also, there is a report that red ginseng promotes the brain activity and enhances the mental activity itself.

However, simply taking ginseng or red ginseng itself is not enough to improve lowering of concentration or memory. Thus, more effective ginseng compositions have been needed. Now, the study for improving memory by using ginseng is focused on preparing a mixture of herb medicine by adding other herb medicine known to be effective for improving memory to the extract of ginseng or red ginseng.

Korean Patent Publication No. 2002-87787 discloses a composition for improving memory by using hairy root of ginseng. The composition described in the Publication is prepared as follows: hairy root of ginseng obtained by tissue culture is extracted with 70% of alcohol or ethanol; non-saponin ingredients from this extract are removed; and desired ingredients are eluted to give the composition. This composition comprises ginsenosides Rb1, Rb2, Rc, Rd, Re and Rg1, wherein each content of ginsenosides Rb1, Rb2, Rc and Rd is ½ of the content of ginsenoside Rg1. However, this method needs a complicated step of tissue culture of ginseng hairy root, and does not standardize the ingredient content of ginsenosides because each content of ginsenosides Rb1, Rb2, Rc and Rd is less than ½ of the content of ginsenoside Rg1.

### DISCLOSURE OF THE INVENTION

The present inventors have repeated extensive studies to develop a new ginseng composition capable of preventing or improving lowering of concentration and memory, and thus discovered that ginsenoside Rg3, Rg5 or Rk1 can prevent or improve lowering of concentration and memory. Particularly, the present inventors discovered that the ginseng composition of the present invention comprising 10 kinds of ginsenoside, main ingredient of ginseng, whose contents are standardized to more than a certain amount, by specially processing traditional ginseng or red ginseng is more effective for the prevention or improvement of lowering of concentration and memory than conventional ginseng compositions, to complete the present invention.

Thus, an object of the present invention is to provide the extract of processed ginseng or lyophilized product thereof; a ginseng composition additionally comprising powder, extract or lyophilized product of red ginseng or white ginseng; a ginseng composition comprising more than a certain amount of 10 kinds of ginsenoside in ginseng; or a composition for the prevention or improvement of lowering concentration and memory capability comprising ginsenoside Rg3, Rg5 or Rk1.

Another object of the present invention is to provide health care products for the prevention or improvement of lowering of concentration and memory capability comprising the above composition.

Another object of the present invention is to provide a use of the above composition to prevent or improve lowering of concentration and memory capability

Another object of the present invention is to provide a method of prevention or improvement of lowering of concentration and memory capability by administering a therapeutically effective amount of the above composition.

Another object of the present invention is to provide a method for preparing a ginseng composition for the prevention or improvement of lowering of concentration and memory capability by mixing and extracting ginseng etc.; or agents for the prevention or improvement of lowering of concentration and memory capability by mixing 10 kinds of ginsenoside in ginseng with a certain ratio.

### BRIEF DESCRIPTION OF THE DRAWINGS

FIG. 1 is a graph showing the present composition effect to space study ability.

### BEST MODE FOR CARRYING OUT THE INVENTION

To accomplish the objects described above, the present invention provides a ginseng composition equally comprising more than a certain amount of ginsenosides Rb1, Rb2, Rc, Rd, Re, Rf and Rg1 and ginsenosides Rg3, Rg5 and Rk1 .

The present invention also provides the extract of processed ginseng obtained by steaming treated ginseng at the temperature under 110° C. for 0.5˜15 hr, wherein the treated ginseng was obtained by treating ginseng with acid at 50˜80° C., or lyophilized product thereof.

The present invention also provides a ginseng composition comprising, i) the extract of processed ginseng obtained by steaming treated ginseng at the temperature under 110° C. for 0.5˜15 hr, wherein the treated ginseng was obtained by treating ginseng with acid at 50˜80° C., or lyophilized product thereof, and ii) powder, extract, or lyophilized product of red ginseng or white ginseng, with the ratio of 0.1˜10:0.1˜10.

The present invention also provides a use of a ginseng composition comprising ginsenosides Rb1, Rb2, Rc, Rd, Re, Rf and Rg1 and ginsenosides Rg3, Rg5 and Rk1; the extract of processed ginseng obtained by steaming treated ginseng at the temperature under 110° C. for 0.5˜15 hr, wherein the treated ginseng was obtained by treating ginseng with acid at 50˜80° C., or lyophilized product thereof; and a ginseng composition additionally comprising powder, extract, or lyophilized product of red ginseng or white ginseng thereto.

The present invention also provides a method for the prevention or improvement of lowering of the concentration and memory capability by administrating a ginseng composition comprising ginsenosides Rb1, Rb2, Rc, Rd, Re, Rf and Rg1 and ginsenosides Rg3, Rg5 and Rk1; the extract of processed ginseng obtained by steaming treated ginseng at the temperature under 110° C. for 0.5˜15 hr, wherein the treated ginseng was obtained by treating ginseng with acid at 50˜80° C., or lyophilized product thereof; and a ginseng composition additionally comprising powder, extract, or lyophilized product of red ginseng or white ginseng thereto.

The present invention also provides a method for preparing agents for the prevention or improvement of lowering of the concentration and memory capability by steaming treated ginseng at the temperature under 110° C. for 0.5˜15 hr, wherein the treated ginseng was obtained by treating ginseng with acid at 50˜80° C.; additionally mixing powder, extract, or lyophilized product of red ginseng or white ginseng thereto; or mixing ginsenosides Rb1, Rb2, Rc, Rd, Re, Rf and Rg1 and ginsenosides Rg3, Rg5 and Rk1 with a certain ratio.

For example, the ginseng composition of the present invention can be prepared by mixing i) the extract or lyophilized product prepared by extracting processed ginseng with water or common organic solvent such as C₁₋₄ alcohol, or mixing solvent thereof, wherein the processed ginseng is prepared by two steps of: treating ginseng with acid at 50˜80° C. (1^{st} step) and steaming the treated ginseng of 1^{st} step at the temperature under 110° C. for 0.5˜15 hr (2^{nd} step) with ii) powder of red ginseng or white ginseng; extract thereof extracted with water or common organic solvent such as C₁₋₄ alcohol, or mixing solvent thereof; or lyophilized product thereof, with the ratio of 0.1˜10:0.1˜10, preferable 1:0.1˜10, more preferable 1:0.1˜5, particularly preferable 1:0.1˜3.

In the present ginseng composition, preferably, the above lyophilized product of processed ginseng is properly mixed with powder of red ginseng or white ginseng, and the mixing ratio is controlled to the above ratio.

The present invention also provides a ginseng composition comprising i) ginsenosides Rb1, Rb2, Rc, Rd, Re, Rf and Rg1, and ii) ginsenosides Rg3, Rg5 and Rk1, with the ratio of 0.1˜10:0.1˜10, preferable 1:0.1˜10, more preferably 1:0.1˜5, particularly preferable 1:0.1˜2.

For example, the above composition can be prepared by controlling the mixing ratio of extract or lyophilized product of processed ginseng; and powder, extract or lyophilized product of red ginseng or white ginseng, so the content ratio of ginsenosides Rb1, Rb2, Rc, Rd, Re, Rf and Rg1; and ginsenosides Rg3, Rg5 and Rk1 can be 0.1˜10:0.1˜10, wherein the contents of ginsenosides are respectively measured from processed ginseng, red ginseng or white ginseng according to the following examples.

If the temperature and time of the above process, and the content ratio of processed ginseng, red ginseng, or white ginseng are lower than the lowest limit, the upward reaction of each ginsenoside is lowered, and then the aiming effects cannot be achieved. Also, if the above content ratio exceeds the highest limit, it is not desirable either since the upward reaction of the composition may be lowered, and the yield of process goes down.

In the present invention, ginseng can be selected from the group comprising Panax ginseng, P. japonicum, P. quinquefolium, P. notoginseng, P. trifolium, and P. pseudoginseng, without limitation, and used by root, stem, leaf, or herb.

In the present invention, the acid which can be used in the 1^{st} step of the above processing method is not particularly limited so long as the acid can cause substitution of the substituent located at 20^{th} carbon of the ginsenoside of ginseng, but preferably, acetic acid. In case of using acetic acid, the concentration of acetic acid is not particularly limited, but may be 20˜100%. Particularly, it is preferable to use acetic acid because the boiling point of acetic acid is about 107° C., and so it can be removed in the steaming process without additional removal process.

In the acid treatment of the 1^{st} step, it is preferable for the steaming temperature to be about 50˜80° C., more preferably 65˜75° C., since the substitution by acid can be promoted in the temperature range. Also, it is preferable to treat ginseng at 70° C. with acid for 0.1˜10 hr, more preferable 1˜5 hr, particularly preferable 3 hr.

In the present invention, the processed ginseng is prepared by steaming the ginseng treated in 1^{st} step at the temperature under 110° C., preferably at 50˜107° C., for 0.5˜15 hr. In case of the processing method described in Korean Patent No. 96-17670, there is a practical drawback that the range of temperature should be maintained at 120˜180° C., which lowers economic efficiency. The present invention is much more convenient than the above patent method, and can increase the contents of ginsenosides Rg3, Rg5 and Rk1with high yield because the ginseng in the present invention is steamed at the temperature under 110° C., preferably at 50˜107° C., for 0.5˜15 hr, preferably 0.5˜8 hr, more preferably 1˜3 hr.

In the present invention, the extract or lyophilized product thereof used for the present composition may be prepared by conventional methods such as broth extraction or sonication with using solvents like water, C₁₋₄ alcohol such as methanol, ethanol, propanol, butanol, etc., or mixed solvents thereof, after the above 2^{nd} step.

The composition of the present invention can remarkably prevent or improve lowering of concentration and memory as shown in the following experimental example.

Also, the present invention provides a composition for preventing or improving lowering of concentration and memory comprising ginsenosides Rg3, Rg5 and Rk1.

The composition of the present invention can be prepared according to conventional methods in the pharmaceutical field into conventional pharmaceutical preparations, for example, solution such as drinks, syrup and capsule, by mixing with pharmaceutically acceptable carrier, excipient, etc.; and administered orally or parenterally. Preferably, the composition of the present invention may be orally administered in drink before and/or after the meal for quick effect.

Capsule and solution comprising the composition of the present invention may be used as medicine or health care products. Here, “health care products” mean food products prepared and processed in the form of tablet, capsule, powder, granule, solution, pill, etc., by using material or ingredients having useful function to the human body.

The composition of the present invention is appropriately administered according to the extent of absorption of active ingredients into the body; excretion rate; age, weight, sex, and condition of patient; severity of treated disease, etc. However, generally, in solution, it is preferable to administer the present composition 1˜3 times a day, 0.5˜10 ml each. In other preparations, an appropriate amount based on the above dose for solution can be administered orally.

Hereinafter, the present invention will be described in more detail with reference to the following examples, but the scope of the present invention should not be construed to be limited thereby in any manner.

### EXAMPLES

A: Preparation of Processed Ginseng

1. Acetic Acid Treatment Process

The steaming instrument [Seogang ENG (Inc.), Korea], concentrator (EYELA, Japan), lyophilizer [Ilshinwrap (Inc.), Korea] used for preparing processed ginseng was owned by the Material Development Team of UNIGEN, Inc. Raw ginseng of 4-years-roots (Keumsan) was used as raw material for processing. Also, to compare the contents of ginsenosides Rg3 and Rg5, besides the raw ginseng of 4-years-roots, red ginseng, white ginseng, white tail ginseng, and raw ginseng of 5-years-roots (Keumsan) were purchased and used.

To estimate optimum concentration for acetic acid treatment and optimum treating method, 100 g of raw ginseng of 4-years-roots was quantified, and put into each of two plastic containers, and 1.5 L of 50% of acetic acid mixed with anhydrous acetic acid and water at the rate of 1:1, and 1.5 L of 100% of anhydrous acetic acid not mixed with water were put into each of the plastic container. One plastic container among the two plastic containers containing acetic acid was heated in water-bath at 70° C. for 3 hr, and the other plastic container was left at the room temperature for 2, 4, 6, 8, 10, 24 and 48 hr without heating.

2. Steaming of Ginseng and Preparation of the Extract

The processed ginseng prepared in the above step A.1. was steamed and extracted for removal of acetic acid, addition of sugar, and hydrolysis. To measure the optimal temperature and time, the raw ginseng treated with acetic acid was steamed under the temperature and time of 120° C. (8 hr), 100° C. (3hr), 80° C. (8 hr), and 80° C. (3 hr), to result in Samples 9˜12.

The processed ginseng was extracted with 70% of ethanol at 80° C. for 6 hr, and then extracted at 45° C. for about 5 hr.

To reduce the pumping effect due to the rising of temperature and decompression, the extracts, which are reactants with high viscosity (65˜70 Brix), were diluted with warm water to lower the viscosity to about 24 Brix, and then the reactants were cooled at −70° C. for 2 days. When the cooling of the reactants was completed, the reactants were lyophilized at −70° C. and 10 mtorr for 2 days.

3. Contents Analysis

To analyze 10 kinds of ginsenoside in reactant of the present invention, the analysis which can analyze 10 kinds of ginsenoside additionally containing ginsenosides Rg3, Rg5 and Rk1 at once, is developed by Unigen Inc., contrary to conventional analysis which only analyzes ginsenosides Rb1, Rb2, Rc, Rd, Re, Rf and Rg1.

The HITACHI system (pump; L-7100, detector; L-7455, interface; D-7000, column oven; L-7300, automatic feeder; L-7200) was used as HPLC for analysis of the reactants. The condition of analysis was as follows: Capcell PAK C18 (5 μm), 3.0*75 mm was used as stationary phase; the condition of mobile phase was gradient control with solvent A of acetonitril, and solvent B of Water; the flow rate was 0.5 ml/min; the total time of isolation was 110 min; the temperature of column oven was 40° C.; the dosage of sample was 10 μl; and the sample was detected by UV detector at 203 nm. Ginsenosides Rb1, Rb2, Rc, Rd, Re, Rf and Rg1were isolated within 60 min, and Rg3, Rg5 and Rk1 were isolated after 70 min.

The samples for analysis of the processed ginseng extract prepared in the above step A. 2. were prepared with methanol to the concentration of 2 mg/ml. The standard samples of ginsenosides were prepared in the concentration of 0.2 mg/ml. Both the standard samples and raw material samples treated with acetic acid were put into automatic feeder, and analyzed.

Raw ginseng of 4-years-roots was processed by using the above acetic acid treatment method according to the conditions in Table 1 (samples 1˜8) below, and the results thereof were analyzed as follows: in case of no heating treatment, Rg3 and Rg5 were hardly formed, and in case the heating treatment was done at 70° C. for 3 hr, Rg3 and Rg5 were formed in the 50% of acetic acid reactant and 100% of acetic acid reactant, respectively. Here, 50% of acetic acid was proven to be the optimal concentration of acetic acid since it is advantageous to minimize the concentration of acetic acid as reaction solvent considering up-scale mass manufacturing process. Also, heating treatment was decided as the treating method.

TABLE 1
Change of ginsenoside content according to pretreatment of acetic acid and
treating method

| Sample | Rb1 | Rb2 | Rc | Rd | Re | Rf | Rg1 | (R)-Rg3 | (S)-Rg3 | Rg5 | Rk1 |
|---|---|---|---|---|---|---|---|---|---|---|---|
| 1 | 13.40 | 4.95 | 6.10 | 2.69 | 9.24 | 2.07 | 3.87 | 0.17 | — | — | — |
| 2 | 20.19 | 7.27 | 6.69 | 4.08 | 15.64 | 2.98 | 7.78 | — | — | — | — |
| 3 | 22.85 | 8.82 | 7.97 | 4.86 | 17.26 | 3.17 | 9.28 | — | — | — | — |
| 4 | 23.61 | 9.44 | 7.82 | 4.91 | 16.90 | 3.46 | 9.28 | — | — | — | — |
| 5 | 23.50 | 9.01 | 8.41 | 4.96 | 17.09 | 3.46 | 9.30 | — | — | — | — |
| 6 | 24.44 | 9.41 | 12.86 | 5.11 | 16.81 | 3.47 | 9.25 | — | — | — | — |
| 7 | 0.46 | — | — | — | — | — | — | 6.75 | 3.47 | 10.45 | 3.24 |
| 8 | 1.25 | — | — | — | — | — | — | 5.46 | 3.84 | 11.78 | 3.77 |

Sample 1: No retting in acetic acid;
Sample 2: Retting in 50% of acetic acid for 2 hr;
Sample 3: Retting in 50% of acetic acid for 4 hr;
Sample 4: Retting in 50% of acetic acid for 6 hr;
Sample 5: Retting in 50% of acetic acid for 8 hr;
Sample 6: Retting in 50% of acetic acid for 10 hr;
Sample 7: Heating in 50% of acetic acid at 70° C., for 3 hr;
Sample 8: Heating in 100% of acetic acid at 70° C., for 3 hr

Comparing the contents of ginsenosides according to the steaming process, relatively high contents of ginsenosides Rg3 and Rg5 were shown at the condition of 120° C. for 8 hr and 100° C. for 3 hr. And, the acetic acid was completely removed at 100° C. In terms of economic efficiency, the temperature and time should be minimum. Therefore, the steaming temperature of 100° C. and the steaming time of 3 hr were assessed to be optimal in the process (Table 2).

TABLE 2
Change of ginsenoside content according to the steaming process

| Sample | Rb1 | Rb2 | Rc | Rd | Re | Rf | Rg1 | (R)-Rg3 | (S)-Rg3 | Rg5 | Rk1 |
|---|---|---|---|---|---|---|---|---|---|---|---|
| 9 | 0.35 | — | — | — | — | 0.72 | — | 15.39 | 13.43 | 29.26 | 12.38 |
| 10 | 0.43 | — | 0.53 | — | — | 0.04 | — | 17.35 | 15.37 | 31.33 | 11.89 |
| 11 | 0.32 | — | 0.42 | — | — | 0.01 | 0.10 | 7.33 | 6.21 | 8.42 | 3.15 |
| 12 | 0.18 | — | 0.40 | — | 0.12 | 0.07 | 0.05 | 9.17 | 7.62 | 9.68 | 4.73 |

Sample 9: Treating with 50% of acetic acid (heating at 70° C. for 3 hr), and then steaming at 120° C. for 8 hr;
Sample 10: Treating with 50% of acetic acid (heating at 70° C. for 3 hr), and then steaming at 100° C. for 3 hr;
Sample 10: Treating with 50% of acetic acid (heating at 70° C. for 3 hr), and then steaming at 80° C. for 3 hr;
Sample 10: Treating with 50% of acetic acid (heating at 70° C. for 3 hr), and then steaming at 80° C. for 8 hr

The contents of ginsenosides according to the kind of ginseng showed similar for all red ginseng, and raw ginseng (Table 3).

TABLE 3
Change of ginsenoside content according to the kind of ginseng (mg/g)

| Sample | Rb1 | Rb2 | Rc | Rd | Re | Rf | Rg1 | (R)-Rg3 | (S)-Rg3 | Rg5 | Rk1 |
|---|---|---|---|---|---|---|---|---|---|---|---|
| 13 | 8.14 | 25.38 | 25.31 | 19.28 | 5.74 | 5.59 | 11.21 | 1.58 | 0.64 | 1.09 | 0.35 |
| 14 | 0.24 | — | — | — | — | — | — | 18.34 | 16.28 | 32.78 | 13.72 |
| 15 | 7.88 | 15.04 | 13.05 | 12.32 | 2.67 | 4.61 | 7.88 | 1.28 | 0.22 | 0.17 | 0.05 |
| 16 | 0.45 | — | — | — | — | — | — | 12.15 | 10.28 | 21.83 | 7.14 |
| 17 | 8.63 | 10.83 | 5.36 | 1.94 | 2.19 | 0.39 | 0.95 | 1.60 | 0.05 | 0.05 | 0.02 |
| 18 | 0.35 | — | — | — | — | — | — | 13.39 | 11.74 | 24.05 | 9.36 |
| 19 | 36.42 | 34.91 | 26.59 | 9.84 | 14.57 | 2.38 | 5.26 | 6.86 | — | 1.55 | 0.48 |
| 20 | 0.69 | — | — | — | — | — | — | 18.15 | 15.28 | 29.78 | 11.24 |

Sample 13: Red ginseng;
Sample 14: Treating red ginseng with 50% of acetic acid (heating at 70° C. for 3 hr), and then steaming at 100° C. for 3 hr;
Sample 15: White ginseng;
Sample 16: Treating white ginseng with 50% of acetic acid (heating at 70° C. for 3 hr), and then steaming at 100° C. for 3 hr;
Sample 17: White tail ginseng;
Sample 18: Treating white tail ginseng with 50% of acetic acid (heating at 70° C. for 3 hr), and then steaming at 100° C. for 3 hr;
Sample 19: Raw ginseng of 5-years-roots;
Sample 20: Treating raw ginseng of 5-years-roots with 50% of acetic acid (heating at 70° C. for 3 hr), and then steaming at 100° C. for 3 hr

In short, the method for preparing particular ginsenoside by using enzyme among the prior patent and preparation methods to increase the contents of ginsenosides Rg3 and Rg5 is not easily applicable to mass manufacturing process because the unit production cost is high, and the manufacturing process steps are complicated. Also, in the method inducing hydrolysis of ginsenosides under high temperature and pressure, it is difficult to establish the condition of high temperature and pressure.

In that sense, the present invention has an advantage in preparing the present composition to contain high content of ginsenosides by using solvent such as acetic acid without establishing the condition of high temperature and pressure. Also, the manufacturing process of the present composition is simpler than others, and thus it is easily applicable to up-scale mass manufacturing process. And, in the recovery and disposal of acetic acid, the process of the present invention is not using and separating a large amount of acetic acid, but is heating the raw material soaked in a certain amount of 50% of acetic acid. Also, used acetic acid is not disposed and can be reused in the state, and so the present invention complies with the environmental requirement under the MSDS.

B: Preparation of a Composition of the Present Invention

Based on the above analysis result, a composition of the present invention was prepared by mixing with ginseng processed by the above processing method; and extract or lyophilized product of red ginseng or white ginseng according to the conventional method to regularly contain the content of 10 kinds of ginsenoside.

### Composition Example 1

25 g of lyophilized powder of the processed ginseng of Sample 14 and 75 g of powder of red ginseng extract were mixed to obtain Composition Example 1. The contents of ginsenosides Rb1, Rb2, Rc, Rd, Re, Rf, and Rg1 and ginsenosides Rg3, Rg5 and Rk1 of the above composition were 6.2, 19.0, 19.0, 14.5, 4.3, 4.2, 8.4 mg/g and 10.4, 9.0, 3.7 mg/g, respectively.

### Composition Example 2

40 g of lyophilized powder of the processed ginseng of Sample 16 and 60 g of powder of white ginseng extract were mixed to obtain Composition Example 2. The contents of ginsenosides Rb1, Rb2, Rc, Rd, Re, Rf and Rg1 and ginsenosides Rg3, Rg5 and Rk1 of the above composition were 4.9, 9.0, 7.8, 7.4, 1.6, 2.8, 4.7 mg/g and 9.8, 8.8, 2.9 mg/g, respectively.

### Composition Example 3

50 g of lyophilized powder of the processed ginseng of Sample 18 and 50 g of powder of white ginseng tail extract were mixed to obtain Composition Example 3. The contents of ginsenosides Rb1, Rb2, Rc, Rd,. Re, Rf and Rg1 and ginsenosides Rg3, Rg5 and Rk1 of the above composition were 4.5, 5.4, 2.7, 1.0, 1.1, 0.2. 0.5 mg/g and 13.4, 12.1, 4.7 mg/g, respectively.

### Composition Example 4

80 g of lyophilized powder of the processed ginseng of Sample 20 and 20 g of powder of white ginseng extract were mixed to obtain Composition Example 4. The contents of ginsenosides Rb1, Rb2, Rc, Rd, Re, Rf and Rg1 and ginsenosides Rg3, Rg5 and Rk1of the above composition were 7.8, 7.0, 5.3, 2.0, 2.9, 0.5. 1.1 mg/g and 28.1, 24.1, 9.1 mg/g, respectively.

### Composition Example 5

50 g of lyophilized powder of the processed ginseng of Sample 20 and 50 g of powder of white ginseng extract were mixed to obtain Composition Example 5. The contents of ginsenosides Rb1, Rb2, Rc, Rd, Re, Rf and Rg1 and ginsenosides Rg3, Rg5 and Rk1 of the above composition were 18.6, 17.5, 13.3, 4.9, 7.3, 1.2, 2.6 mg/g and 21.1, 15.7, 5.9 mg/g, respectively.

### Composition Example 6

25 g of lyophilized powder of the processed ginseng of Sample 20 and 75 g of powder of white ginseng extract were mixed to obtain Composition Example 6. The contents of ginsenosides Rb1, Rb2, Rc, Rd, Re, Rf and Rg1 and ginsenosides Rg3, Rg5 and Rk1 of the above composition were 27.5, 26.2, 19.9, 7.4, 10.9, 1.8, 3.9 mg/g and 13.5, 8.6, 3.2 mg/g, respectively.

### Composition Example 7

20 g of lyophilized powder of the processed ginseng of Sample 20 and 80 g of powder of white ginseng extract were mixed to obtain Composition Example 7. The contents of ginsenosides Rb1, Rb2, Rc, Rd, Re, Rf and Rg1 and ginsenosides Rg3, Rg5 and Rk1 of the above composition were 29.3, 27.9, 21.3, 7.9, 11.7, 1.9, 4.2 mg/g and 12.2, 7.2, 2.6mg/g, respectively.

### Composition Example 8

10 g of lyophilized powder of the processed ginseng of Sample 20 and 90 g of powder of white ginseng extract were mixed to obtain Composition Example 8. The contents of ginsenosides Rb1, Rb2, Rc, Rd, Re, Rf and Rg1 and ginsenosides Rg3, Rg5 and Rk1 of the above composition were 32.8, 31.4, 23.9, 8.9, 13.1, 2.1, 4.7 mg/g and 9.5, 4.4, 1.6mg/g, respectively.

### Composition Example 9

50 g of lyophilized powder of the processed ginseng of Sample 20 and 75 g of powder of red ginseng extract were mixed to obtain Composition Example 9. The contents of ginsenosides Rb1, Rb2, Rc, Rd, Re, Rf and Rg1 and ginsenosides Rg3, Rg5 and Rk1 of the above composition were 4.4, 12.7, 12.7, 9.6, 2.9, 2.8, 5.6 mg/g and 17.9, 15.4, 5.8 mg/g, respectively.

### EXPERIMENTAL EXAMPLE

1. Division of Experimental Animal and Groups

500 g of SD rats in the age of 40 weeks were used as experimental animal in this experiment. The groups were divided into normal group, treating group of red ginseng extract (RG), composition group of Example 1 (extract group comprising 10 kinds of ginsenosides Rb1, Rb2, Rc, Rd, Re, Rf, Rg1, Rg3, Rg5, and Rk1; UG10S), and group comprising 3 kinds of ginsenosides Rg3, Rg5, and Rk1 as active ingredients (UG3). Each sample of 300 mg/kg was dissolved in water and administered to the experimental animal for 2 months, to be used for the following experiment.

2. Morris Water Maze Test

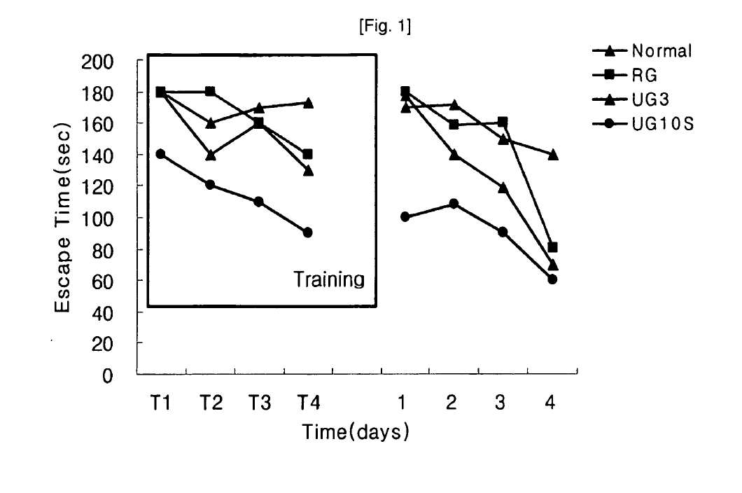

The water tank used as water maze was shaped like cylinder having the diameter of 180 cm and the height of 40 cm, and filled with water of 22±2° C. up to the height of 30 cm. Then, each figured cue was attached to two (2) places on the wall around the water tank. Escape support was plastic post of round shape having the diameter of 15 cm, and placed 3 cm below from the water surface. The water maze was divided into four equal parts, northeast (NE), northwest (NW), southeast (SE) and southwest (SW). Escape support was placed at the center part of the northeast quartering circle. The starting point was 2 places, each of which was used as the starting place at two different starts in real time. The experimental animals were trained twice for 180 sec a day, and the total training period is 4 days. After 2 days' rest, the test result was calculated at the same condition as training. The results were shown in the following FIG. 1. The test for space study ability was to test whether the rats remember previous studied contents when they study for several days, rest for a while, and repeat previous study. In the present results, the group treated with red ginseng arrived at the aimed place more quickly than normal rats (this corresponds to the reported result). Also, the composition group of Example 1 (UG10S) and UG3 group arrived at the aimed place more quickly than normal rats, and arrived at the aimed place more quickly than the group treated with red ginseng which has been already reported. This shows that the composition group of the present invention remembers longer than the normal group and the group treated with red ginseng. Also, during the training, the composition group of the present invention arrived at the aimed place more quickly than the normal group and the group treated with red ginseng. This shows that the learning ability to a certain study of the composition group of Example 1 (UG10) and UG3 group is faster than that the normal group and the group treated with red ginseng. Also, during early training, the composition group of the present invention arrived at the aimed place more quickly than the normal group and the group treated with red ginseng, which shows increase of study effect to new content since we conducted the experiment after treating the sample for 2 months. Such different result in the animal test can be very significant.

Also, as known in FIG. 1, the arriving time to escape support of the composition group of Example 1 (UG10) and UG3 group was much shorter than the normal group. Also, comparing the composition group with the group treated with red ginseng (RG) whose effect has been already reported, though it was during trailing, the composition group of Example 1 arrived at the escape support more quickly, and in re-test after stopping study for 2 days, the composition group of Example 1 arrived more quickly. This shows that the memory about space study of the composition group of Example 1 (UG10) and UG3 group were kept longer than that of the group treated with red ginseng (RG).

Based on the above test result, it was confirmed that the effect of space study ability of the composition of the present invention comprising ginsenosides Rg3, Rg5 and Rk1, or 10 kinds of ginsenosides is superior to that of the normal group. Also, it was confirmed that the effect of space study ability of the composition of the present invention is superior to that of the red ginseng extract used as test control.

### Formulation Example 1

### Preparation of Solution

| Composition of Composition Example 1 | 20 | g |
|---|---|---|
| Sugar | 10 | g |
| Isomerized sugar | 10 | g |
| Smell of lemon | proper quantity | |
| Total amount after adding purified water | 100 | ml |

The above-mentioned ingredients were mixed according to conventional preparation method for solution, and sterilized to give solution.

### Formulation Example 2

### Preparation of Solution

| Composition of Composition Example 3 | 30 | g |
|---|---|---|
| Sugar | 10 | g |
| Isomerized sugar | 10 | g |
| Smell of lemon | proper quantity | |
| Total amount after adding purified water | 100 | ml |

The above-mentioned ingredients were mixed according to conventional preparation method for solution, and sterilized to give solution.

### Formulation Example 3

### Preparation of Capsule

| | |
|---|---|
| Composition of Composition Example 6 | 500 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to conventional preparation method for capsule to give capsule.

### Formulation Example 4

### Preparation of Capsule

| | |
|---|---|
| Lyophilized powder of Sample 14 | 35 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to conventional preparation method for capsule to give capsule.

### Formulation Example 5

### Preparation of Capsule

| | |
|---|---|
| Ginsenoside Rg3 | 10 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to conventional preparation method for capsule to give capsule.

### Formulation Example 6

### Preparation of Drink

A mixture of 6 weight % of composition of Composition Example 9, 5 weight % of fructose, 0.1 weight % of citric acid, and a proper amount of lemon flavor is prepared, and further purified water was added thereto, to give drink.

### INDUSTRLIAL APPLICABILITY

As known from the above, in the present invention, a new ginseng composition can be prepared by equally comprising 10 kinds of ginsenoside in ginseng, which can induce the maximum synergic effect due to each ginsenoside. Also, the only product in the world having the highest level can be prepared by standardizing the contents of each ginsenoside. The above composition or Ginsenosides Rg3, Rg5 and Rk1 can effectively prevent and improve lowering of concentration and memory capability.

1. A extract of processed ginseng obtained by steaming treated ginseng at the temperature under 110° C. for 0.5˜15 hr, wherein the treated ginseng was obtained by treating ginseng with acid at 50˜80° C., or lyophilized product thereof.

2. A ginseng composition comprising, i) the extract of processed ginseng obtained by steaming treated ginseng at the temperature under 110° C. for 0.5˜15 hr, wherein the treated ginseng was obtained by treating ginseng with acid at 50˜80° C., or lyophilized product thereof, and ii) powder, extract, or lyophilized product of red ginseng or white ginseng, with the ratio of 0.1˜10:0.1˜10.

3. The ginseng composition according to claim 2, wherein the content ratio is 1:0.1˜0.

4. The ginseng composition according to claim 1 or 2, wherein the extract is extract of water, C₁₋₄ alcohol, or mixing solvent thereof.

5. The ginseng composition according to claim 1 or 2, characterized in that the acid is acetic acid.

6. The ginseng composition for the prevention or improvement of lowering of the concentration and memory capability according to any of claims 1 to 3.

7. A composition for the prevention or improvement of lowering of the concentration and memory capability comprising ginsenosides Rg3, Rg5 and/or Rk1 as active ingredient.

8. A composition comprising, i) ginsenosides Rb1, Rb2, Rc, Rd, Re, Rf and Rg1, and ii) ginsenosides Rg3, Rg5 and Rk1, with the content ratio of 0.1˜10:0.1˜10.

9. The composition according to claim 8, wherein the content ratio is 1:0.1˜10.

10. The composition for the prevention or improvement of lowering of the concentration and memory capability according to claim 8 or 9.

11. Health care products comprising the composition according to any of claim 1, 2, 7 or 8.

12. (canceled)

13. (canceled)

14. (canceled)

15. (canceled)

16. (canceled)

17. (canceled)

18. (canceled)

19. (canceled)

20. A method of preventing or improving lowering of the concentration and memory capability, comprising administering a therapeutically effective amount of extract of processed ginseng obtained by steaming treated ginseng at the temperature under 110° C. for 0.5˜15 hr, wherein the treated ginseng was obtained by treating ginseng with acid at 50˜80° C., or lyophilized product thereof, to mammal.

21. A method of preventing or improving lowering of the concentration and memory capability, comprising administering a therapeutically effective amount of ginseng composition comprising, i) the extract of processed ginseng obtained by steaming treated ginseng at the temperature under 110° C. for 0.5˜15 hr, wherein the treated ginseng was obtained by treating ginseng with acid at 50˜80° C., or lyophilized product thereof, and ii) powder, extract, or lyophilized product of red ginseng or white ginseng, with the ratio of 0.1˜10:0.1˜10, to mammal.

22. The method according to claim 21, wherein the content ratio is 1:0.1˜10.

23. The method according to claim 20 or 21, wherein the extract is extract of water, C₁₋₄ alcohol, or mixing solvent thereof.

24. The method according to claim 20 or 21, characterized in that the acid is acetic acid.

25. A method of preventing or improving lowering of the concentration and memory capability, comprising administering a therapeutically effective amount of ginsenosides Rg3, Rg5 and/or Rk1 to mammal.

26. A method of preventing or improving lowering of the concentration and memory capability, comprising administering a therapeutically effective amount of composition comprising, i) ginsenosides Rb1, Rb2, Rc, Rd, Re, Rf and Rg1, and ii) ginsenosides Rg3, Rg5 and Rk1, with the content ratio of 0.1˜10:0.1˜10, to mammal.

27. The method according to claim 26, wherein the content ratio is 1:0.1˜10.

28. A method for preparing extract of processed ginseng or lyophilized product thereof for the prevention or improvement of lowering of the concentration and memory capability by steaming treated ginseng at the temperature under 110° C. for 0.5˜15 hr, wherein the treated ginseng was obtained by treating ginseng with acid at 50˜80° C.

29. A method for preparing ginseng composition for the prevention or improvement of lowering of the concentration and memory capability by mixing i) the extract of processed ginseng obtained by steaming treated ginseng at the temperature under 110° C. for 0.5˜15 hr, wherein the treated ginseng was obtained by treating ginseng with acid at 50˜80° C., or lyophilized product thereof, and ii) powder, extract, or lyophilized product of red ginseng or white ginseng, with the ratio of 0.1˜10:0.1˜10.

30. The method according to claim 29, wherein the content ratio is 1:0.1˜10.

31. The method according to claim 28 or 29, wherein the extract is extract of water, C₁₋₄ alcohol, or mixing solvent thereof.

32. The method according to claim 28 or 29, characterized in that the acid is acetic acid.

33. A method for preparing agents for preventing or improving lowering of the concentration and memory capability by mixing i) ginsenosides Rb1, Rb2, Rc, Rd, Re, Rf and Rg1, and ii) ginsenosides Rg3, Rg5 and Rk1, with the content ratio of 0.110:0.1˜10.

34. The method according to claim 33, wherein the content ratio is 1:0.1˜10.
